(19) United States
(12) Patent Application Publication
(10) Pub. No.: US 2002/0111514 A1
(43) Pub. Date: Aug. 15, 2002

(54) Polyaldehyde reaction products

(76) Inventors: Wei Li, Mason, OH (US); Jianhua Mao, West Chester, OH (US); James A. McCaulley, Ringoes, NJ (US)

Correspondence Address: COGNIS CORPORATION, 2500 RENAISSANCE BLVD., SUITE 200, GULPH MILLS, PA, 19406

(21) Appl. No.: 10/013,566

(22) Filed: Dec. 11, 2001

Related U.S. Application Data
(60) Provisional application No. 60/256,375, filed on Dec. 18, 2000.
(60) Provisional application No. 60/309,240, filed on Jul. 31, 2001.

(51) Int. Cl. C07C 323/33; C07C 217/04; C07C 47/198

(52) U.S. Cl. 564/502; 568/41; 568/497

(57) ABSTRACT

Reaction products of the reaction between reactants comprised of the following:

A) at least one compound of formula I below

RX(EO)ₙ(PO)ₘ(BO)ₚH (I)

wherein R is a substituted or unsubstituted, saturated or unsaturated, organic group having from 4 to 36 carbon atoms; X is —O—, —S—, or —NR₂— where R₂ is hydrogen or a C₁-C₈ alkyl group; n is a number from 0 to 100; m is a number from 0 to 50; and p is a number from 0 to 50; provided that the sum of n, m, and p is at least 1; and

B) a polyaldehyde of formula II below

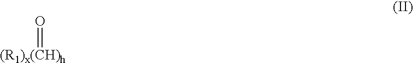

where R₁ is a C₁-C₃₀ straight or branched chain optionally substituted alkyl or alkenyl group, an optionally substituted aromatic group, or an optionally substituted cycloalkyl or cycloalkenyl group; x is 0 or 1; and h is a number of at least two.

### CROSS REFERENCE TO RELATED APPLICATIONS

This application claims the benefit of copending provisional application serial No. 60/256,375, filed on Dec. 18, 2000, and provisional application serial No. 60/309,240 filed on Jul. 31, 2001; the entire contents of each of which are incorporated herein by reference.

### FIELD OF THE INVENTION

This invention relates to reaction products useful as low foaming surfactants and as defoaming agents in aqueous and nonaqueous liquid compositions.

### BACKGROUND OF THE INVENTION

Aqueous cleaning compositions exhibit a tendency toward foaming since they contain surface active agents such as soaps, and synthetic detergents. In many instances, such cleaning compositions produce excessive foam and the user must add substances known as anti-foaming agents or defoamers. Some defoamers such as silicones tend to interfere with the function of the cleaning compositions in that unwanted residues are left after the cleaners are wiped off, while others are environmentally unacceptable because they are not biodegradable.

Alkyl polyglycosides are a class of nonionic surfactants that exhibit significantly higher foaming profiles than other nonionic surfactants, such as alcohol ethoxylates. In fact, the foaming tendencies of alkyl polyglycosides more closely resemble those of anionic surfactants, such as alcohol sulfates, than the foaming tendencies of other nonionic surfactants. The higher foaming tendency makes the use of alkyl polyglycosides alone undesirable for many applications, e.g. cleaning-in-place for food processing plants, high-pressure spray cleaning, bottle washing, floor cleaners, and automatic dish washing, wherein high levels of foam interfere with the cleaning and rinsing operation and reduce the efficiency of the operation.

Low foam nonionics, such as EO/PO block copolymers, can be used to reduce the foaming properties of alkyl polyglycosides and anionic surfactants, but these materials have undesirable properties, e.g. low biodegradability, relatively high aquatic toxicity, and poor caustic compatibility.

Accordingly, there is a need for the development of defoamers that do not interfere with the cleaning ability of aqueous cleaning compositions and that are biodegradable, exhibit low aquatic toxicity, and good caustic compatibility.

There is also a need for defoamers for nonaqueous compositions.

In addition, there is a continuing need for low foaming surfactants for use in both aqueous and nonaqueous compositions.

### SUMMARY OF TE INVENTION

This invention relates to the reaction products of

A) at least one compound of formula I

RX(EO)ₙ(PO)ₘ(BO)ₚH (I)

wherein R is a substituted or unsubstituted, saturated or unsaturated, organic group having from 4 to 36 carbon atoms; X is —O—, —S—, or —NR₂— where R₂ is hydrogen or a C₁-C₈ alkyl group; n is a number from 0 to 100, e.g. from 1 to 100; m is a number from 0 to 50, e.g. from 1 to 50; and p is a number of from 0 to 50, e.g. from 1 to 50; provided that the sum of n, m, and p is at least 1, and more preferably at least 2; and

B) a polyaldehyde of formula II below:

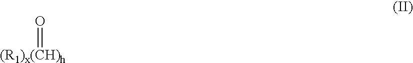

where R₁ is a C₁-C₃₀ straight or branched chain optionally substituted alkyl or alkenyl group, an optionally substituted aromatic group, or an optionally substituted cycloalkyl or cycloalkenyl group; x is 0 or 1; and h is a number of at least two.

The reaction products of component A) and B) will include one or more compounds of formula III below

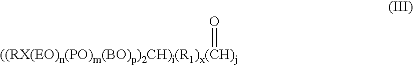

where i+j=h in formula II, and i is at least one.

### DESCRIPTION OF THE PREFERRED EMBODIMENTS

Other than in the operating examples, or where otherwise indicated, all numbers expressing quantities of ingredients or reaction conditions used herein are to be understood as modified in all instances by the term “about”. Also, throughout this description, unless expressly stated to the contrary; percent, “parts” of, and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description or of generation in situ by chemical reactions specified in the description, and does not necessarily preclude other chemical interactions among the constituents of a mixture once mixed.

In the formula I reactant, formula I compounds wherein the sum of n, m, and p is at least 2, especially at least 3, and more especially at least 4, are preferred.

In the compounds of formulas I and III, it is understood that EO stands for the residue of ethylene oxide, PO stands for the residue of propylene oxide, and BO stands for the residue of butylene oxide. Also, in the compounds of formula I, the EO, PO, and BO groups, when present, can be in any order with respect to the RX group, and can be in blocks and/or in random distribution, although the alkoxide groups present are preferably present in the order shown in formulas I and III.

The substituents that can be present on the substituted R groups in formula I can be single or multiple substituents such as one or more halogen substituents, for example Cl, Fl, I, and Br; a sulfur functionality such as a mercaptan or thio group; a nitrogen functionality such as an amine or amide functionality; an alcohol functionality, a silicon functionality, e.g., a siloxane; an ether functionality, e.g., a C₁-C₆ alkoxy group; or any combination thereof

As stated above, the R group in formula I can be any substituted or unsubstituted, saturated or unsaturated organic moiety having from 4 to 36 carbon atoms. Thus, when the R group is an aliphatic group, the group can be a linear or branched alkyl group, a linear or branched alkenyl or alkynyl group, a saturated carbocyclic moiety, an unsaturated carbocyclic moiety having one or more multiple bonds, a saturated heterocyclic moiety, an unsaturated heterocyclic moiety having one or more multiple bonds, a substituted linear or branched alkyl group, a substituted linear or branched alkenyl or alkynyl group, a substituted saturated carbocyclic moiety, a substituted unsaturated carbocyclic moiety having one or more multiple bonds, a substituted saturated heterocyclic moiety, and a substituted unsaturated heterocyclic moiety having one or more multiple bonds. Examples of the above include but are not limited to an alkyl group having from 4 to 22 carbon atoms, an alkenyl group having from 4 to 22 carbon atoms, an alkynyl group having from 4 to 22 carbon atoms, an aryl group having from 6 to 26 carbon atoms, e.g., phenyl, naphthyl, etc., and an arenyl group having from 7 to 36 carbon atoms. Arenyl groups are alkyl-substituted aromatic radicals having a free valence at an alkyl carbon atom such as a benzylic group. Alkyl groups having from 4 to 12 carbon atoms are preferred, and alkyl groups having from 8 to 10 carbon atoms are most preferred. The degree of ethoxylation is preferably from 2 to 50 with the most preferred being from 4 to about 50 while the degree of propoxylation and butoxylation can vary from 0 to about 50, e.g. from 1 to 10. The presence of, and degree of, propoxylation and/or butoxylation will be determined by the desired degree of solubility or miscibility in aqueous and/or nonaqueous compositions. The solubility and miscibility will ultimately be determined by such factors as the number of carbon atoms in R and the relative amounts of EO, PO, and BO therein, as well as similar factors with respect to the polyaldehyde of formula II.

In the polyaldehyde of formula II, the R₁ group is preferably an unsubstituted alkyl group containing from 1 to 8 carbon atoms, and h is preferably a number of from 2 to 10, more preferably from 2 to 4.

When the R₁ group is an aromatic group, e.g. phenyl, naphthyl, anthryl, etc., or a cycloalkyl or cycloalkenyl group, the group can be unsubstituted or substituted, for example substituted with one or more of the following; C₁-C₆ alkyl groups, halogen groups such as chlorine, bromine, iodine, or fluorine, C₁-C₆ alkoxy groups, and the like.

The

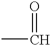

groups can be attached to any available carbon atom in the R₁ group. For example, when the substituents on the substituted aromatic, cycloalkyl, or cycloalkenyl group include one or more C₁-C₆ alkyl groups, the

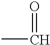

groups can be attached to a ring structure and/or to the C₁-C₆ alkyl groups.

Examples of polyaldehydes of Formula II include, but are not limited to, glyoxal, 1,2-benzenedicarboxaldehyde, 3-formyl-benzenepropanal, 3-octendial, glutaconaldehyde, 1,1,5-pentanetricarboxaldehyde, and 2,7-naphthalenedicarboxaldehyde.

In the compounds of formula III, it is preferred that j=o and therefore i=h in formula II, i.e. all of the aldehyde groups in formula II have reacted.

The reaction between components A) and B) can be carried out in an inert hydrocarbon solvent at a temperature in the range of from about 20 to 125° C., preferably at a temperature of less that 100° C., e.g. from 20 to 80° C., and more preferably at reflux temperature at ambient pressure. An inert atmosphere such as a nitrogen atmosphere can be used. The reaction proceeds well in the presence of an acidic catalyst, e.g. paratoluene-sulfonic acid. Other liquid acidic catalysts can also be employed, such as HNO₃, H₂SO₄, H₃PO4, etc. Also, solid polymeric acidic catalysts, e.g. NAFION® resin (Du Pont), AMBERLY® ST 15 (Aldrich chemicals) preferably prewashed with water, can be employed, but the yields of product from such solid polymeric acidic catalysts are usually not as good as those obtained with p-toluene sulfonic acid. After the reaction has proceeded to completion, usually after 4 to 15 hours, the acid catalyst is neutralized, and the reaction mixture is filtered to produce the filtrate product.

Unreacted excess component A) when present can be removed from the reaction product, e.g. by thin film evaporation.

The reaction products of the invention can be used as low foaming surfactants in both aqueous and nonaqeous compositions in surfactant-effective amounts, usually from 0.1 to 10% by weight, preferably from 1 to 5% by weight, based on the weight of the composition.

These reaction products can also be used in the above quantities as defoaming agents for aqueous and nonaqueous compositions, and are particularly useful in minimizing or eliminating foaming in aqueous compositions containing high foaming surfactants, such as alkyl polyglycosides and anionic surfactants such as alcohol sulfates.

These reaction products can be used in aqueous cleaning compositions, emulsion polymer latex compositions such as latex paints, in inks, in adhesives, in metal working compositions, and in other aqueous and nonaqueous compositions in which surfactants and/or defoaming agents are advantageously present.

The reaction products of the invention are biodegradable, contain no organic solvents, and do not adversely affect the detergency of other surfactants that may be present in compositions in which they are used since they are themselves surfactants.

The invention will be illustrated but not limited to the following example.

### EXAMPLE

Preparation of Glyoxal Bis (Dibutyl Acetal)

A mixture containing 3.62 g (25.0 mmole) of glyoxal (40 wt. % solution in water), 34.23 g (100 mmole) of POE (5.5) hexanol, and 80 mg (0.8 mmole) of MeSO₃H acid in 20 mL of toluene was heated at reflux for 14.0 hours with continuous azeotropic removal of water. The cooled mixture was washed in successive order with saturated aqueous sodium bicarbonate (20 mLs) and saturated brine (20 mLs). The washed organic layer was then dried over anhydrous sodium sulfate and filtered. Mw result of SEC indicated that peak value Mw was 1353, close to theoretical Mw 1398.

What is claimed is:

1. A compound of formula III 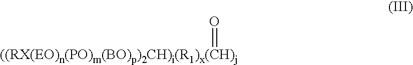
wherein R is an organic group having from 4 to 36 carbon atoms; X is —O—, —S—, or —NR₂— where R₂ is hydrogen or a C₁-C₄ alkyl group; R₁ is a C₁-C₃₀ straight or branched chain optionally substituted alkyl or alkenyl group, an optionally substituted aromatic group, or an optionally substituted cycloalkyl or cycloalkenyl group; n is a number of from 0 to 100; m is a number of from 0 to 50; p is a number of from 0 to 50; EO is ethyleneoxy; PO is propyleneoxy; BO is butyleneoxy; x is 0 or 1; i+j is a number of at least 2; provided that the sum of n, m, and p is at least one, and that i is at least one.

2. The compound of formula II wherein j=0.

3. The compound of formula III in claim 1 wherein the sum of n, m, and p is at least 2.

4. The compound of formula III in claim 1 wherein X is —O—.

5. The compound of formula III in claim 1 wherein R is an alkyl group containing from 4 to 22 carbon atoms.

6. The compound of formula III in claim 5 wherein R contains from 4 to 12 carbon atoms.

7. The compound of formula III in claim 1 wherein n=4 to about 50.

8. The compound of formula III in claim 1 wherein EO, PO, and BO when present are in the order show.

9. The compound of formula III in claim 1 wherein EO, PO, and BO when present are in block and/or random distribution and are in any order shown with respect to the XR groups.

10. The compound of formula III in claim 1 wherein X is —O—; R is an alkyl group containing from 4 to 22 carbon atoms; n=4 to about 50; and m and p=0.

11. The compound of formula III in claim 10 wherein R₁ is a C₁-C₃₀ straight or branched chain alkyl or alkenyl group.

12. The compound of formula III in claim 1 which is glyoxal bis (dibutyl acetal).

13. The reaction product of the reaction between reactants comprised of the following:
A) at least one compound of formula I below
RX(EO)ₙ(PO)ₘ(BO)ₚH (I)
wherein R is a substituted or unsubstituted, saturated or unsaturated, organic group having from 4 to 36 carbon atoms; X is —O—,—S—, or —NR₂— where R₂ is hydrogen or a C₁-C₈ alkyl group; n is a number from 0 to 100; m is a number from 0 to 50; and p is a number from 0 to 50; provided that the sum of n, m, and p is at least 1; and
B) a polyaldehyde of formula II below 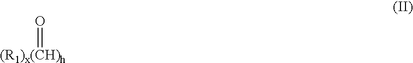
where R₁ is a C₁-C₃₀ straight or branched chain optionally substituted alkyl or alkenyl group, an optionally substituted aromatic group, or an optionally substituted cycloalkyl or cycloalkenyl group; x is 0 or 1; and h is a number of at least two.

14. The reaction product of claim 13 wherein in component A), the sum of n, m, and p is at least two.

15. The reaction product of claim 13 wherein in component A), X is —O—.

16. The reaction product of claim 13 wherein said reaction is acid catalyzed.

17. The reaction product of claim 13 wherein in component A), R is an alkyl group containing from 4 to 22 carbon atoms.

18. The reaction product of claim 17 wherein R contains from 4 to 12 carbon atoms.

19. The reaction product of claim 13 wherein in component A), n=4 to about 50.

20. The reaction product of claim 13 wherein in component A), EO, PO, and BO when present are in the order shown.

21. The reaction product of claim 13 wherein in component A), X is —O—; R is an alkyl group containing from 4 to 22 carbon atoms; n=4 to about 50; and m and p=0.

22. The reaction product of claim 21 wherein in component B), R₁ is a C₁-C₃₀ straight or branched chain alkyl or alkenyl group.

23. In an aqueous composition, the improvement wherein a surfactant-effective or defoaming-effective quantity of the compound of claim 13 is present therein.

24. The composition of claim 23 wherein the surfactant-effective or defoaming-effective quantity is from about 0.1 to about 10% by weight.

25. The composition of claim 23 wherein the composition is a latex paint composition.

26. In an aqueous composition containing an alkyl polyglycoside and/or an alcohol sulfate, the improvement wherein a defoaming-effective quantity of the compound of claim 13 is present therein.

27. In a nonaqueous composition, the improvement wherein a surfactant-effective or defoaming-effective quantity of the compound of claim 13 is present therein.

28. The nonaqueous liquid composition of claim 27 wherein the composition is an ink, an adhesive, or a metal working composition.

29. The nonaqueous liquid composition of claim 27 wherein the surfactant-effective quantity is from 0.1 to about 10% by weight.

30. A process for the preparation of the reaction product of claim 13 comprising the steps of
I) reacting the at least one compound of formula I
RX(EO)ₙ(PO)ₘ(BO)ₚH (I)
with an aldehyde of formula II 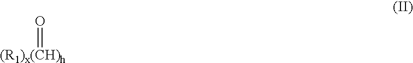
in the presence of an acidic catalyst at a temperature of from about 20 to about 125° C.; and
II) isolating the reaction product from the resulting reaction mixture.

31. The process of claim 30 wherein water is continually removed from the reaction mixture in step A) by azeotropic distillation with an organic solvent.

32. The process of claim 31 wherein the organic solvent is a hydrocarbon solvent.

33. The process of claim 30 wherein in step A) the reaction temperature is less than 100° C.

34. The process of claim 30 wherein the reaction temperature is the reflux temperature of the mixture.

35. The process of claim 30 wherein in step A) the acid catalyst is paratoluenesulfonic acid.
